(19) United States
(12) Patent Application Publication
(10) Pub. No.: US 2012/0253471 A1
(43) Pub. Date: Oct. 4, 2012

(54) Variable Length Airway Stent Graft With One-Way Valves

(75) Inventors: Irene Tully, Galway (IE); Brian Kelly, Galway (IE)

(73) Assignee: Medtronic Vascular, Inc., Santa Rosa, CA (US)

(21) Appl. No.: 13/079,297

(22) Filed: Apr. 4, 2011

(51) Int. Cl. A61F 2/04

(52) U.S. Cl. 623/23.65

(57) ABSTRACT

An airway stent graft includes a plurality of openings through the surface of the graft material and one-way valves at the openings to permit air from outside of the stent graft to enter a lumen thereof during exhalation and to prevent air from exiting the lumen through the openings during inhalation. The stent graft may be of variable length such that the stent graft may be cut to the desired length in vivo. A delivery system includes a cutter assembly to cut graft material of the stent at the desired location in vivo.

### FIELD OF THE INVENTION

Embodiments hereof relate to a variable length airway stent graft with one-way valves used in the treatment of chronic obstructive pulmonary disease, and delivery systems and methods for delivering and implanting such a graft.

### BACKGROUND OF THE INVENTION

Chronic obstructive pulmonary disease (COPD) refers to a group of diseases that cause airflow blockage and related respiratory problems. It includes emphysema, chronic bronchitis, and in some cases asthma. COPD is a major cause of disability, and it is the fourth leading cause of death in the United States. More than 12 million people are currently diagnosed with COPD. Many more people may have the disease without being diagnosed.

Those inflicted with COPD face disabilities due to the limited pulmonary function. Usually, individuals afflicted by COPD also face loss in skeletal muscle strength and an inability to perform common daily activities. Often, those patients desiring treatment for COPD seek a physician at a point where the disease is advanced. Since the damage to the lungs is irreversible, there is little hope of recovery. Most times, the physician cannot reverse the effects of the disease but can only offer symptomatic treatment and advice to halt the progression of the disease.

The primary function of the lungs is to permit the exchange of two gasses by removing carbon dioxide from arterial blood and replacing it with oxygen. To facilitate this exchange, the lungs provide a blood-gas interface. The oxygen and carbon dioxide move between inhaled gas (air) and blood by diffusion. This diffusion is possible since the blood is delivered to one side of the blood-gas interface via small blood vessels (capillaries). The capillaries are wrapped around numerous air sacs called alveoli which function as the blood-gas interface. A typical human lung contains about 300 million alveoli.

Air is brought to the other side of this blood-gas interface by a natural respiratory airway, consisting of branching tubes which become narrower, shorter, and more numerous as they penetrate deeper into the lung. Specifically, the airway begins with the trachea which branches into the left and right bronchi which divide into lobar, then segmental bronchi. Ultimately, the branching continues down to the terminal bronchioles which lead to the alveoli. Plates of cartilage may be found as part of the walls throughout most of the airway from the trachea to the bronchi. The cartilage plates become less prevalent as the airways branch. Eventually, in the last generations of the bronchi, the cartilage plates are found only at the branching points. The bronchi and bronchioles may be distinguished as the bronchus lies proximal to the last plate of cartilage found along the airway, while the bronchiole lies distal to the last plate of cartilage. The bronchioles are the smallest airways that do not contain alveoli. The function of the bronchi and bronchioles is to provide conducting airways that lead air to and from the gas-blood interface. However, these conducting airways do not take part in gas exchange because they do not contain alveoli. Rather, the gas exchange takes place in the alveoli which are found in the distalmost end of the airways.

The mechanics of breathing include the lungs, the rib cage, the diaphragm and abdominal wall. During inspiration, inspiratory muscles contract increasing the volume of the chest cavity. As a result of the expansion of the chest cavity, the pleural pressure, the pressure within the chest cavity, becomes sub-atmospheric. Consequently, air flows into the lungs and the lungs expand. During unforced expiration, the inspiratory muscles relax and the lungs begin to recoil and reduce in size. The lungs recoil because they contain elastic fibers that allow for expansion as the lungs inflate and relaxation as the lungs deflate with each breath. This characteristic is called elastic recoil. The recoil of the lungs causes alveolar pressure to exceed atmospheric pressure causing air to flow out of the lungs and deflate the lungs. If the ability of the lungs to recoil is damaged, the lungs cannot contract and reduce in size from their inflated state. As a result, the lungs cannot evacuate all of the inspired air.

In addition to elastic recoil, the lung's elastic fibers also assist in keeping small airways open during the exhalation cycle. This effect is also known as “tethering” of the airways. Tethering is desirable since small airways do not contain cartilage that would otherwise provide structural rigidity for these airways. Without tethering, and in the absence of structural rigidity, the small airways collapse during exhalation and prevent air from exiting thereby trapping air within the lung.

Emphysema is characterized by irreversible biochemical destruction of the alveolar walls that contain the elastic fibers, called elastin, described above. The destruction of the alveolar walls results in a dual problem of reduction of elastic recoil and the loss of tethering of the airways. Unfortunately for the individual suffering from emphysema, these two problems combine to result in extreme hyperinflation (air trapping) of the lung and an inability of the person to exhale. In this situation, the individual will be debilitated since the lungs are unable to perform gas exchange at a satisfactory rate.

One further aspect of alveolar wall destruction is that the airflow between neighboring air sacs, known as collateral ventilation or collateral air flow, is markedly increased as when compared to a healthy lung. While alveolar wall destruction decreases resistance to collateral ventilation, the resulting increased collateral ventilation does not benefit the individual since air is still unable to flow into and out of the lungs. Hence, because this trapped air is rich in CO₂, it is of little or no benefit to the individual.

Chronic bronchitis is characterized by excessive mucus production in the bronchial tree. Usually there is a general increase in bulk (hypertrophy) of the large bronchi and chronic inflammatory changes in the small airways. Excessive amounts of mucus are found in the airways and semisolid plugs of this mucus may occlude some small bronchi. Also, the small airways are usually narrowed and show inflammatory changes.

Currently, although there is no cure for COPD, treatment includes bronchodilator drugs, and lung reduction surgery. The bronchodilator drugs relax and widen the air passages thereby reducing the residual volume and increasing gas flow permitting more oxygen to enter the lungs. Yet, bronchodilator drugs are only effective for a short period of time and require repeated application. Moreover, the bronchodilator drugs are only effective in a certain percentage of the population of those diagnosed with COPD. In some cases, patients suffering from COPD are given supplemental oxygen to assist in breathing. Unfortunately, aside from the impracticalities of needing to maintain and transport a source of oxygen for everyday activities, the oxygen is only partially functional and does not eliminate the effects of the COPD. Moreover, patients requiring a supplemental source of oxygen are usually never able to return to functioning without the oxygen.

Lung volume reduction surgery is a procedure that removes portions of the lung that are over-inflated. The portion of the lung that remains has relatively better elastic recoil, providing reduced airway obstruction. The reduced lung volume also improves the efficiency of the respiratory muscles. However, lung reduction surgery is an extremely traumatic procedure which involves opening the chest and thoracic cavity to remove a portion of the lung. As such, the procedure involves an extended recovery period. Hence, the long term benefits of this surgery are still being evaluated.

More recently proposed treatments include the use of devices that employ RF or laser energy to cut, shrink or fuse diseased lung tissue. Another lung volume reduction device utilizes a mechanical structure that is used to roll the lung tissue into a deflated, lower profile mass that is permanently maintained in a compressed condition. As for the type of procedure used, open surgical, minimally invasive and endobronchial approaches have all been proposed. Another proposed device (disclosed in publication no. WO 98/48706) is positioned at a location in the lung to block airflow and isolate a part of the lung.

Accordingly, there is a need in the art for improved methods and devices for treating the debilitating affects of pulmonary diseases, in particular COPD, without the need for risky lung reduction surgery.

### BRIEF SUMMARY OF THE INVENTION

Embodiments hereof are directed to an airway stent graft for use in treating chronic obstructive pulmonary disease. The airway stent graft includes a plurality of stents substantially aligned along a common central axis and graft material coupled to the stents such that the stents and graft material form a hollow, tubular structure including a lumen. A plurality of openings through the graft material include one-way valves to permit air from outside of the stent graft to enter the lumen through the openings during exhalation and to prevent air in the lumen from escaping through the openings during inhalation. When implanted in an airway of a lung with at least some of the openings aligned with branch airways, the one-way valves help alleviate over-inflation of the lung by preventing air from entering the branch airways during inhalation and permitted air to escape the branch airways during exhalation.

Embodiments hereof are also directed to delivery systems for delivering a variable length airway stent graft to a treatment site. The delivery system includes an elongated inner shaft, the stent graft mounted in the delivery system around the inner shaft, and an elongated outer sheath enclosing the stent graft in a radially compressed configuration for delivery to the desired anatomic site. The delivery system further includes a cutter assembly for cutting the graft material in vivo at a desired length.

Embodiments hereof are also directed to a method for delivering and deploying an airway stent graft to an anatomic site. The stent graft is disposed in the delivery system in a radially compressed and longitudinally compressed configuration. The longitudinally compressed configuration is provided by folding graft material between adjacent stents of the stent graft. Upon reaching the anatomic site, the outer sheath of the delivery system is retracted to allow a distal stent portion of the stent graft to radially expand itself against walls of the anatomic site. The delivery system is then retracted to longitudinally extend the graft material between the distal stent portion of the stent graft and a first stent of the plurality of stents proximal to the distal stent portion. During this retraction the first stent is not released from the delivery system. The outer sheath is then retracted again to allow the first stent to radially expand against the walls of the anatomic site. The delivery system is then retracted to longitudinally extend the graft material between the first stent portion and a second stent of the plurality of stents proximal to the first stent, wherein the delivery system is retracted such that the second stent is not released from the outer sheath. The outer sheath is then retracted to allow the second stent to radially expand itself against the walls of the anatomic site. These steps are repeated until the desired length of stent graft is released from the delivery system, either by reaching the proximal end of the stent graft, or by cutting the graft material in vivo at a location distal of the proximal end of the stent graft.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a schematic illustration of a known lung and airways in a human body.

FIG. 2 is a schematic perspective illustration of an embodiment of an airway stent graft in accordance with the invention.

FIG. 3 is a schematic cut-open illustration of a portion of the stent graft of FIG. 2.

FIG. 4 is a schematic side view of a one-way valve in the stent graft of FIG. 2.

FIG. 5 is a schematic illustration of the valve of FIG. 4 as viewed from outside the stent graft.

FIG. 6 is a schematic illustration of the valve of FIG. 4 as viewed from inside the stent graft.

FIG. 7 is a schematic, partial perspective sectional illustration of the valve of FIG. 4, shown in a closed position.

FIG. 8 is a schematic, partial perspective sectional illustration of the valve of FIG. 4, shown in an open position.

FIG. 9 is a schematic illustration of the stent graft of FIG. 2 implanted into an airway of a lung, shown during inhalation.

FIG. 10 is a schematic illustration of the stent graft of FIG. 2 implanted into an airway of a lung, shown during exhalation.

FIGS. 11-23 are schematic illustrations of a delivery system for implanting the stent graft of FIG. 2 into an airway and a method of implanting the stent graft into the airway.

FIG. 24 is a schematic perspective illustration of an embodiment of a cutter shaft and cutter.

FIG. 25 is a schematic cross-sectional illustration of the cutter assembly of FIG. 24.

FIG. 26 is a schematic side view of the cutter assembly of FIG. 24.

FIG. 27 is a schematic side view of an embodiment of an inner shaft and stent stopper.

FIG. 28 is a schematic perspective illustration of the inner shaft and stent stopper of FIG. 27.

FIG. 29 is a schematic front view of the stopper of FIG. 27.

FIG. 30 is a schematic top view of the cutter assembly of FIGS. 24-26 disposed over the inner shaft of FIG. 27 with the tabs of the cutter assembly disposed through the slots of the stent stopper of FIG. 27.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

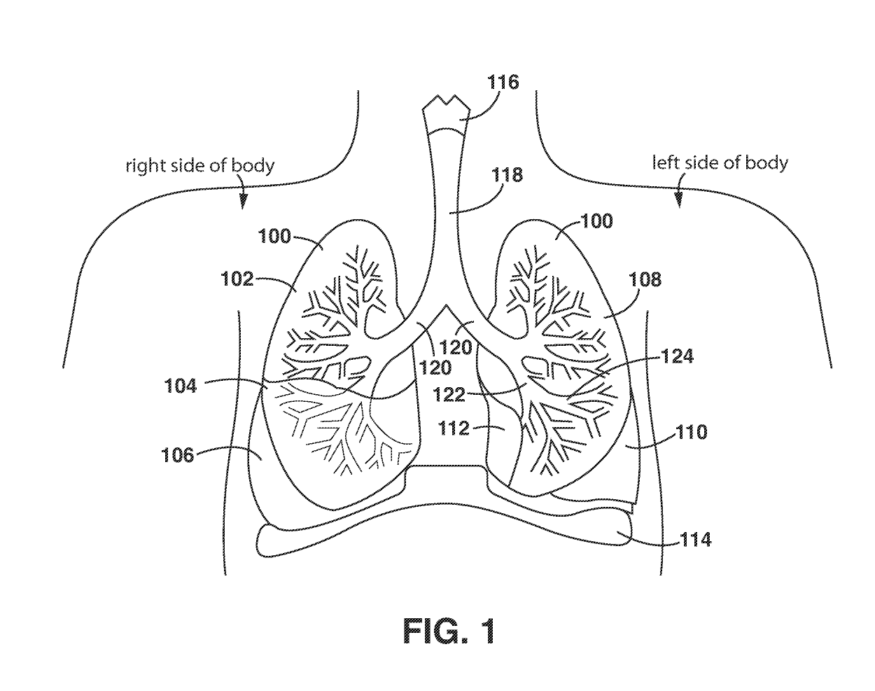

As described briefly in the Background section above and shown in FIG. 1, the human body includes lungs 100 having a right superior lobe 102, a right middle lobe 104, a right inferior lobe 106, a left superior lobe 108, and a left inferior lobe 110. A cardiac notch 112 in the left superior lobe 108 provides space for the heart. As discussed above, the mechanics of inspiration of air involve the diaphragm 114. As a result of the expansion of the chest cavity, the pleural pressure, i.e. the pressure within the chest cavity, becomes sub-atmospheric. Consequently, air flows into the lungs through the larynx 116 and the trachea 118. The trachea 118 divides into two bronchi 120, which each divide further into bronchial tubes 122, segmented bronchi 124, and eventually to alveoli (not shown) where the blood-gas exchange occurs. During unforced expiration, the lungs recoil causing alveolar pressure to exceed atmospheric pressure, thereby causing air to flow out of the lungs in the reverse path as that described above.

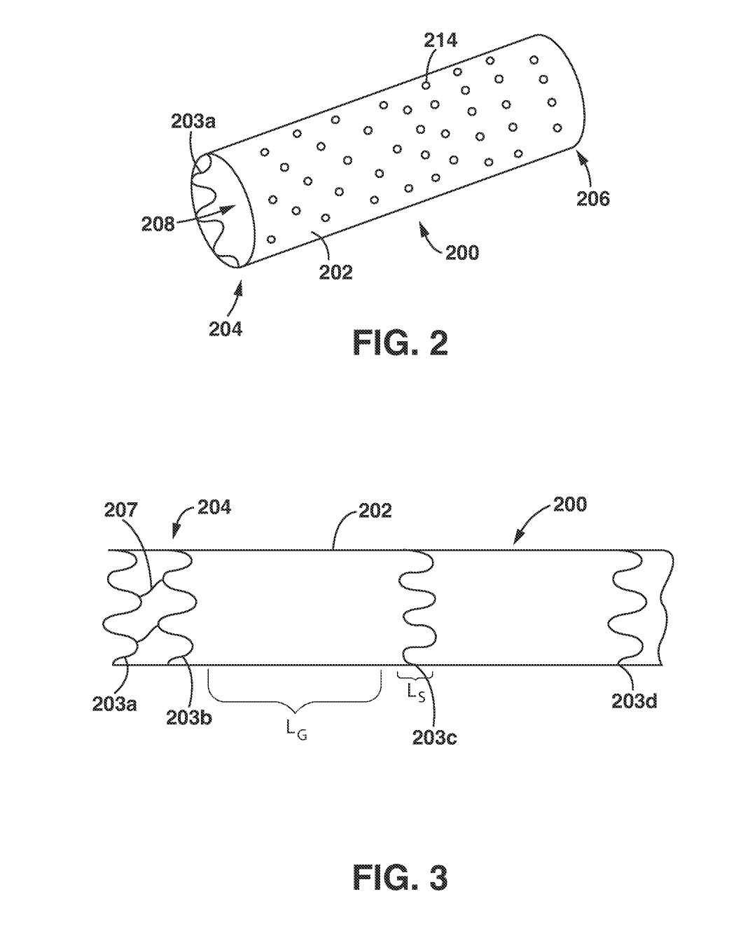

As described above, the lungs of patients with COPD experience reduced ability to recoil such as to evacuate all of the air inspired into the lungs. Further, patients with COPD may experience collapse of small airways due to damage to the lung's elastic fibers. An embodiment of a stent graft 200 shown in FIGS. 2-6 assists in reducing the effects of trapped air due to reduced recoil and collapsed airways.

In particular, the stent graft 200 shown in FIGS. 2-3 in its expanded configuration is a generally tubular configuration including a distal end 204, a proximal end 206, and a lumen 208 disposed therebetween. Graft 200 further includes one-way openings or valves 214 disposed about the periphery of graft 200, as explained in more detail below. Graft 200 may be sized to fit in airways from about 2 mm to about 6 mm in diameter. Accordingly, the outer diameter of graft 200 is sized slightly larger than the airway into which it is to be implanted.

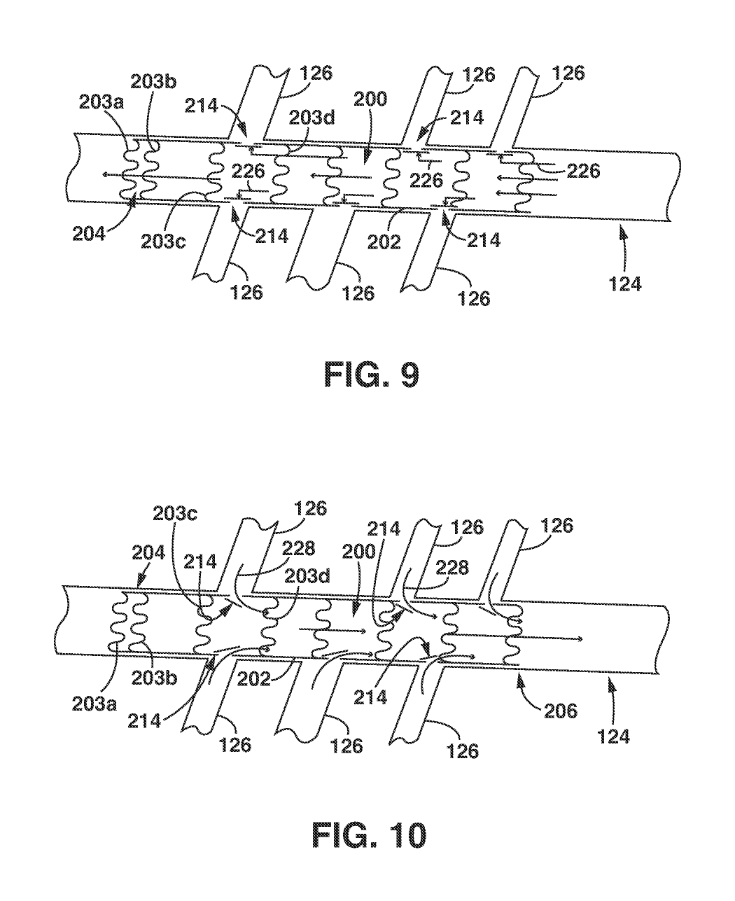

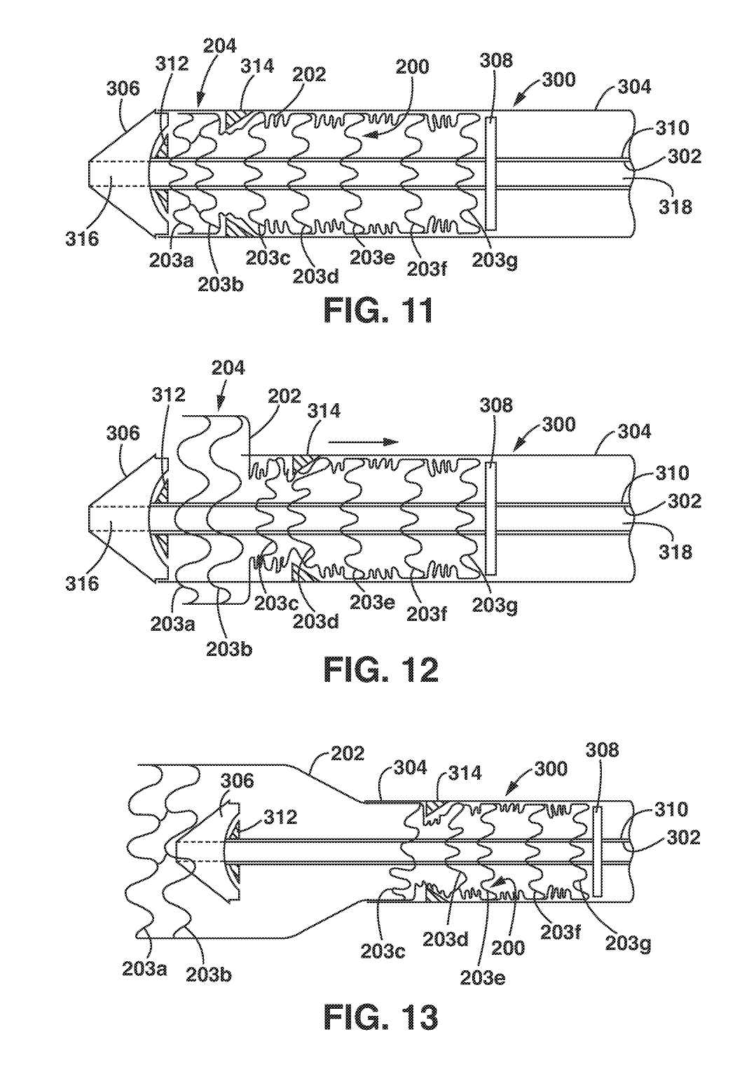

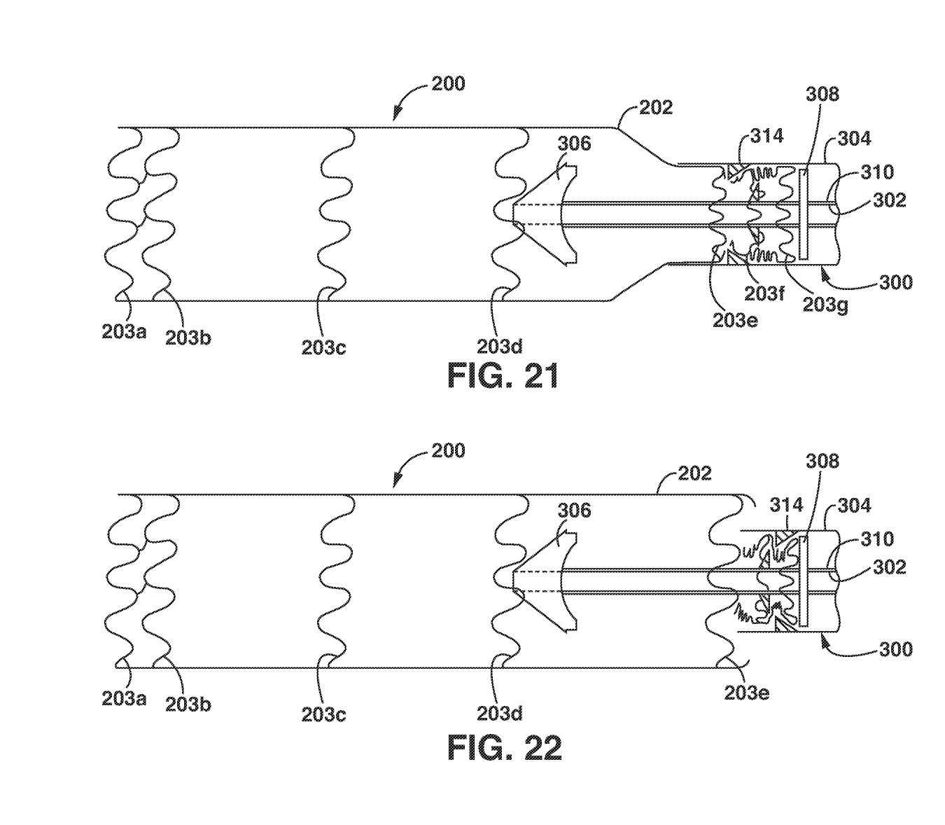

Graft 200 further includes stents 203a, 203b, 203c, 203d coupled to graft material 202, as shown in FIG. 3. Although the stents are referred to by individual reference numerals, they also can be referred to generally as stents 203. Further, four stents 203 are shown in FIG. 2, which shows only a portion of stent graft 200. Accordingly, more stents 203 may be included, as shown for example, in FIGS. 9-23. Further, more or fewer stents 203 may be utilized, depending on the clinical application or other circumstances. Thus, the inclusion of four stents 203 in FIG. 3, or the eight stents 203 shown in FIGS. 9-10, the seven stents 203 in FIGS. 11-22 are merely examples. Stents 203 may be conventional stents known to those skilled in the art and may be made, for example, of stainless steel, “super elastic” titanium-nickel alloy (nitinol) capable of forming stress-induced martensite (SIM), nickel-cobalt-chromium-molybdenum work-hardenable “superalloy,” tantalum, titanium, platinum, gold, silver, palladium, iridium, or other materials known to those skilled in the art. Stents 203 are preferably self-expanding, however, balloon-expandable stents may also be utilized. Graft material 202 may be a generally nonporous, elastic material. Graft material 202 may be, for example and not by way of limitation, a thermoplastic elastomer, silicone, or urethane. Graft material 202 may be attached to stents 203 by stitching, adhesives, or other ways known to those skilled in the art.

In the embodiments shown herein, distal portion 204 of stent graft 200 includes two stents 203a, 203b disposed adjacent to each other and connected to each other with connector elements 207. Those of ordinary skill in the art will understand that stent graft distal portion 204 may include more or fewer stents 203 and, if more than one stent 203 is used, the stents 203 may or may not be connected to each other, and various connecting elements 207 may be used, as known to those of ordinary skill in the art. Stents 203 at distal portion 204 hold stent graft 200 in place during and after the operations to implant graft 200 into the airway, as discussed in more detail below. Accordingly, the number, size, and expansion force of stent(s) 203 at distal portion 204 may be selected to fulfill such a function.

The distance between adjacent stents 203 proximal of the distal portion 204 of stent graft 200, for example, length L_{G} between stents 203b and 203c, may be between three and five times the length of the stents themselves. For example, and not by way of limitation, the length L_{S} of stents 203 may be in the range of 3-6 mm and the length L_{G} may be in the range of 10-30 mm. In another non-limiting example, length L_{S} of stents 203 may be in the range of 5-10 mm and the length L_{G} may be in the range of 15-50 mm.

As shown in FIG. 2, stent graft 200 includes a plurality of one-way openings or valves 214 disposed about the periphery thereof and through graft material 202. One-way valves 214 may be arranged all over the periphery of stent graft 200 such that stent graft 200 need not be perfectly aligned with branches of the airway into which it is inserted, as would be the situation if only a minimal number one-way valves 214 were disposed on graft material 202 of stent graft 200. It would be understood by those skilled in the art that the number of one-way valves through graft material 202 may be varied depending upon the length of stent graft 200, the desired implantation site, the ability to align valves 214 with airway branches, and other factors known to those skilled in the art.

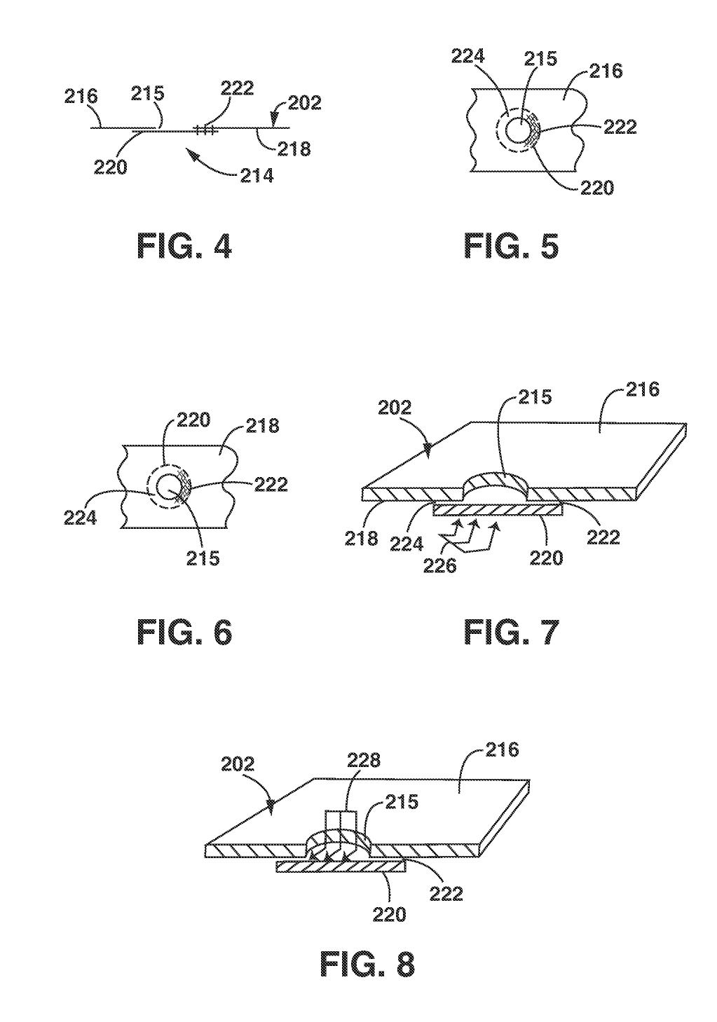

FIGS. 4-8 show an embodiment of one-way valves 214. In this embodiment, valve 214 includes an opening 215 through graft material 202. Graft material 202 includes an outer surface 216 and an inner surface 218. A flap 220 is coupled to graft material 202 and abuts inner surface 218 of graft material 202. Flap 220 is larger than opening 215 and is positioned such that flap 220 covers all of opening 212 when flap is positioned against inner surface 218 of graft material 202. A portion 222 of flap 220 is attached to graft material 202. Portion 222 of flap 220 may be attached to graft material 202 using stitching, adhesive or other attachment methods known to those skilled in the art. Accordingly, a portion 224 of flap 220 is not attached to graft material 202.

FIGS. 9-10 show stent graft 200 implanted into an airway 124 such as a segmented bronchus. Branch airways 126 diverge from airway 124. During inhalation, the pressure discussed in the Background section above forces unattached portion 224 of flap 220 against inner surface 218 of graft material 202, as shown, for example, in FIGS. 7 and 9. With unattached valve portion 224 against inner stent graft surface 218 of graft material, inhaled air cannot pass through one-way valve 214, as depicted by arrows 226, thus preventing air from entering damaged or diseased portions of the lung, as also shown in FIGS. 7 and 9. During exhalation, pressure is relieved and unattached valve portion 224 is separated from inner stent graft surface 218, as shown, for example in FIGS. 8 and 10. Thus, air from damaged or diseased portions of the lung is permitted to escape through valves 214, as depicted by arrows 228 in FIGS. 8 and 10. Further, stents 203 of stent graft 200 keep open the airway into which graft 200 is inserted to further assist air trapped in damaged or diseased portions of the lung to escape.

FIGS. 11-23 illustrate a delivery system 300 for delivery and implantation of stent graft 200 or a similar graft and steps in a method for delivering and implanting stent graft 200 in an airway. For the sake of clarity, the airway into which stent graft 200 is implanted is not shown in FIGS. 11-23. As shown in FIG. 11, delivery system 300 includes an inner shaft 302, a sheath 304, a distal tip 306, and a stent stopper 308. Inner shaft 302 is affixed to tip 306 and extends proximally to a proximal end of delivery system 300 and includes a guidewire lumen 318 that aligns with a guidewire lumen 316 through distal tip 306. A cutter shaft 310 is slidably disposed around inner shaft 302 and has a cutter 312 disposed at a distal end thereof. A wedge 314 is attached to an inner surface of sheath 304. Cutter 312 and wedge 314 cooperate to cut stent graft 200 at a desired length during delivery and implantation into an airway, as will be discussed in more detail below.

Stent graft 200 is loaded into delivery system 300 such that distal portion 204 of stent graft 200 is disposed adjacent to delivery system tip 306. Stent graft 200 is disposed within delivery system 300 in a radially compressed configuration within sheath 304. Further, graft material 202 between stents 203 is folded such that, when in the loaded configuration, stent graft 200 is also in a longitudinally compressed configuration. Wedge 314 of sheath 304 is disposed between stents 203. In the particular embodiment shown, wedge 314 is initially disposed between stents 203b and 203c of stent graft 200, as shown in FIG. 11.

A guidewire (not shown) is navigated through trachea 116, one of the left or right bronchi 120, a bronchial tube 122, and into a segmented bronchus 124. The guidewire is back-loaded into guidewire lumen 316 of delivery system tip 306 and into guidewire lumen 318 of inner shaft 302, as known to those skilled in the art. Delivery system 300 is then advanced over the guidewire to the desired implantation location within an airway such as a segmented bronchus 124.

Upon reaching the desired implantation site, sheath 304 is retracted proximally while inner shaft 302 is held in fixed position with respect to the patient, as shown in FIG. 12. Sheath 304 may be retracted proximally over shaft 302 by methods and devices known to those skilled in the art. Stent stopper 308 prevents stent graft 200 from moving proximally with sheath 304, thereby creating relative movement between sheath 304 and graft 200. Alternatively, similar relative movement may be achieved by holding sheath 304 stationary with respect to the patient while inner shaft 302 is advanced distally. In the step shown in FIG. 12, sheath 304 is retracted only an amount sufficiently to expose distal portion 204 of stent graft 200, thereby permitting stents 203a and 203b of graft 200 to self-expand to a radially expanded configuration. Stents 203a and 203b expand to cause distal portion 204 to abut or approximate an inner surface of the airway (not shown in FIG. 12). Because wedge 314 is attached to sheath 304, wedge 314 has also moved proximally during the relative movement described above such that wedge 314 is moved to a position between stents 203c and 203d of stent graft 200, as shown in FIG. 12.

Next, the entire delivery system 300 is retracted proximally relative to distal portion 204 of stent graft 200 and the airway (not shown), as illustrated in FIG. 13. Sheath 304, inner shaft 302, and stopper 308 are all moved together such that there is minimal relative movement between them. Further, friction between sheath 304 and graft material 202 surrounding stents 203 still within sheath 304 maintains the axial position of the stents 203 within sheath 304 relative to sheath 304. Thus, as delivery system 300 is refracted proximally, graft material 202 disposed between stent 203b and stent 203c unfolds and is straightened, as shown in FIG. 13.

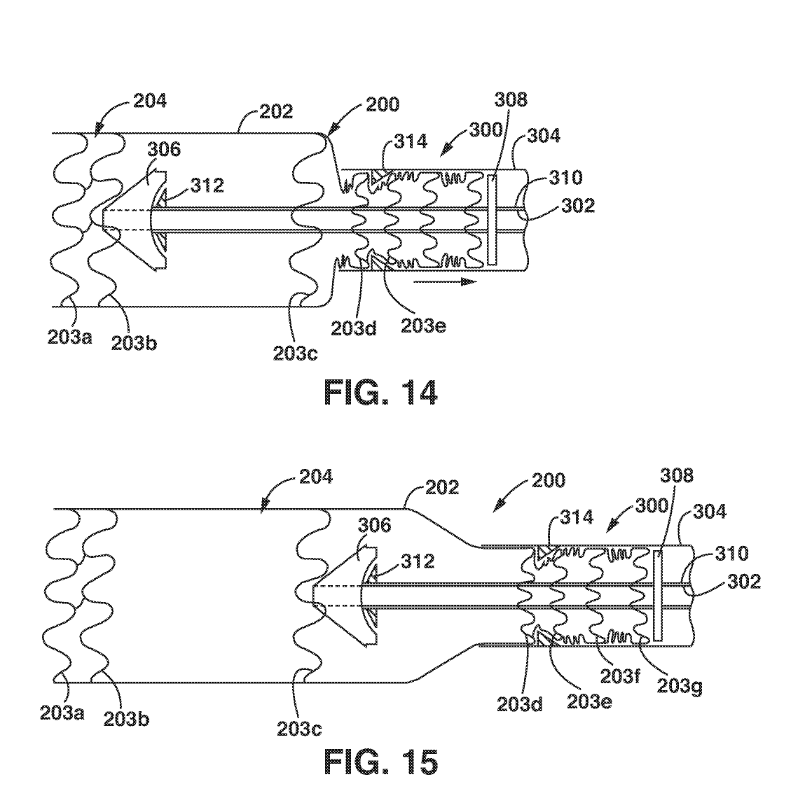

When graft material 202 between stents 203b, 203c has been straightened, sheath 304 is again retracted proximally relative to inner shaft 302, as shown in FIG. 14. As described above, stent stopper 308 prevents stent graft 200 from moving proximally such that sheath 304 releases stent 203c, thereby allowing stent 203c to self-expand and compress the surrounding graft material 202 against the inner wall of the airway.

When stent 203c has self-expanded, delivery system 300 is again retracted proximally, as shown in FIG. 15, and in similar fashion to the step described above with respect to FIG. 13. As described, friction between sheath 304 and graft material 202 surrounding stents 203 still within sheath 304 maintains the axial position of the stents 203 within sheath 304 relative to sheath 304. Thus, as delivery system 300 is refracted proximally, graft material 202 disposed between stent 203c and stent 203d unfolds and is straightened, as shown in FIG. 15.

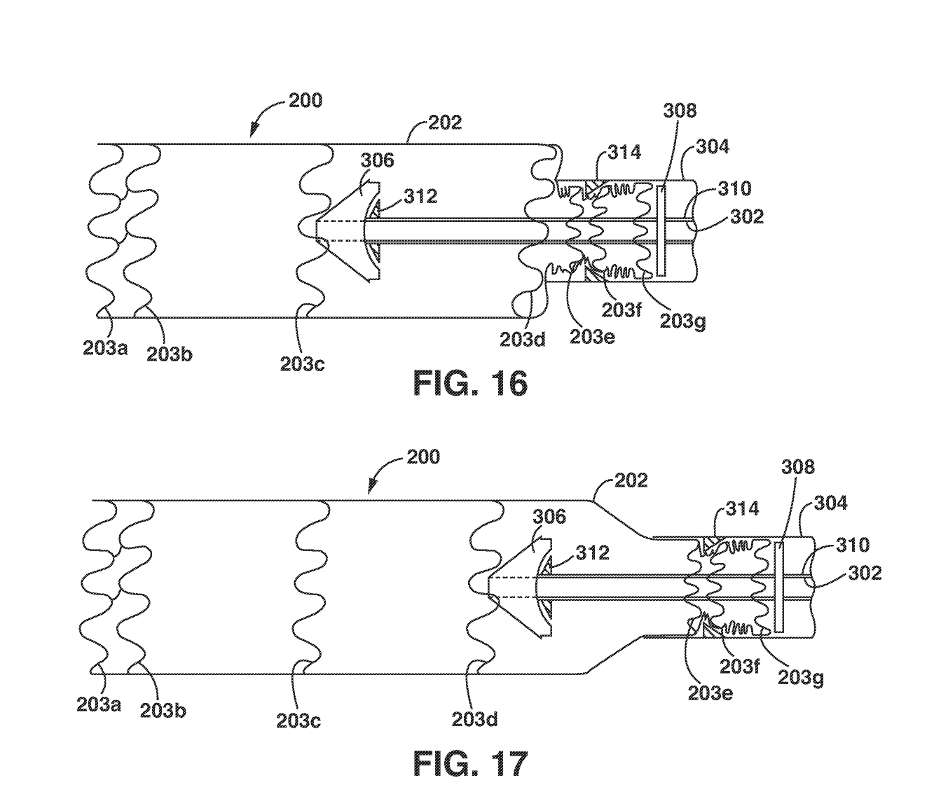

When graft material 202 between stents 203c, 203d has been straightened, sheath 304 is again retracted proximally relative to inner shaft 302, as shown in FIG. 16. As described above, stent stopper 308 prevents graft 200 from moving proximally such that sheath 304 releases stent 203d, thereby allowing stent 203d to self-expand and hold the surrounding graft material 202 against the inner wall of the airway.

When stent 203d has self-expanded, delivery system 300 is again retracted proximally, as shown in FIG. 17, and similar to the steps described above with respect to FIGS. 13 and 15. As described, friction between sheath 304 and graft material 202 surrounding stents 203 still within sheath 304 and sheath 304 maintains the position of the stents 203 within sheath 304 relative to sheath 304. Thus, as delivery system 300 is retracted proximally, graft material 202 disposed between stent 203d and stent 203e unfolds and is straightened, as shown in FIG. 17.

The cutter assembly described herein permits the length of stent graft 200 to be trimmed or adjusted in vivo. Thus, a standard length of stent graft 200 such as the longest length expected to be needed, may be kept in stock and trimmed (i.e., shortened) in vivo depending on the desired final length for a particular patient. Assuming for illustration purposes that a particular procedure/patient requires a length of stent graft 200 spanning and including stents 203a and 203e and the stents 203 therebetween, the cutter assembly is utilized to cut graft material 202 proximal of stent 203e.

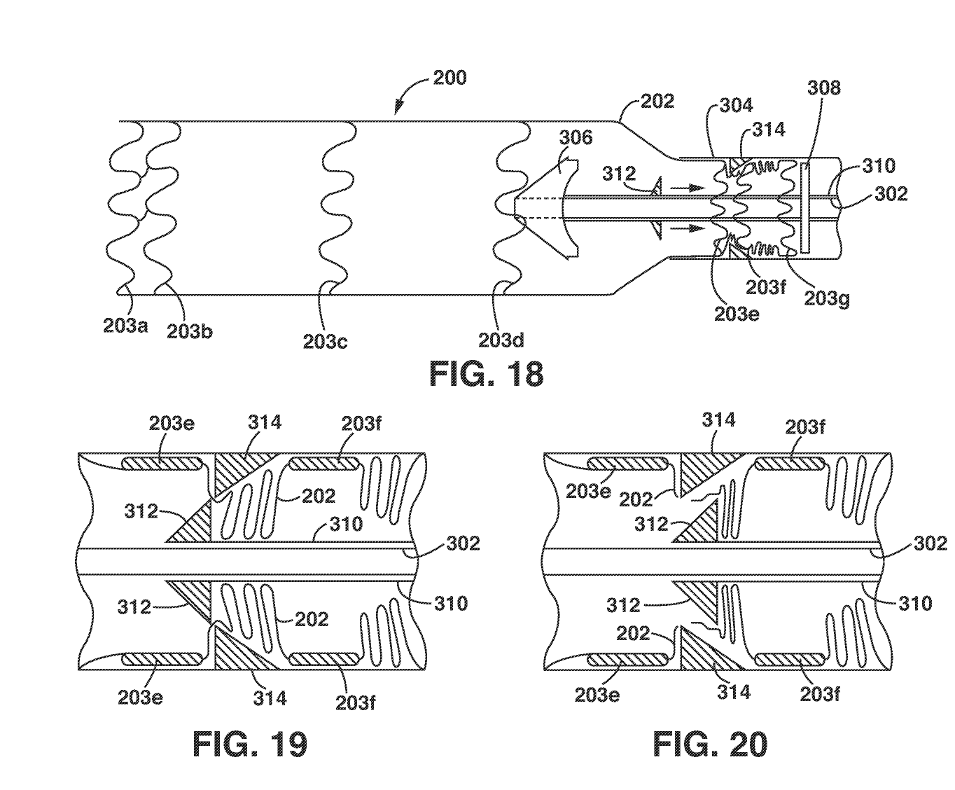

In particular, cutter shaft 310, with cutter 312 attached to a distal end thereof, may be retracted proximally relative to inner shaft 302. FIG. 18 illustrates cutter shaft 310 after retraction thereof has started, but prior to cutter 312 reaching stent 203e. As cutter shaft 310 continues to be retracted proximally, cutter 312 passes wedge 314, and a portion of graft material 202 between stent 203e and stent 203f is captured between cutter 312 and wedge 314, and is cut, as shown in FIGS. 19-21. Those skilled in the art would recognize that the timing of when to retract cutter 312 depends on factors including, but not limited to, the desired length of graft 200, the distance between the distal end of sheath 304 and wedge 314, and the location of wedge 314 relative to stents 203. Further, in this particular embodiment, although cutter 312 is retracted after the graft material 202 between stents 203d and 203e has been straightened, it is recognized that cutter 312 can be retracted to cut graft material 202 between stents 203e and 203f prior to straightening graft material 202 between stents 203d and 203e. However, straightening the graft material first reduces the risk of cutter 312 snagging or inadvertently cutting graft material 202 between stents 203d and 203e rather than between stents 203e and 203f. Further, although a particular embodiment for the cutter assembly is described herein, other devices and methods for adjusting the length of graft 200 in situ may also be utilized.

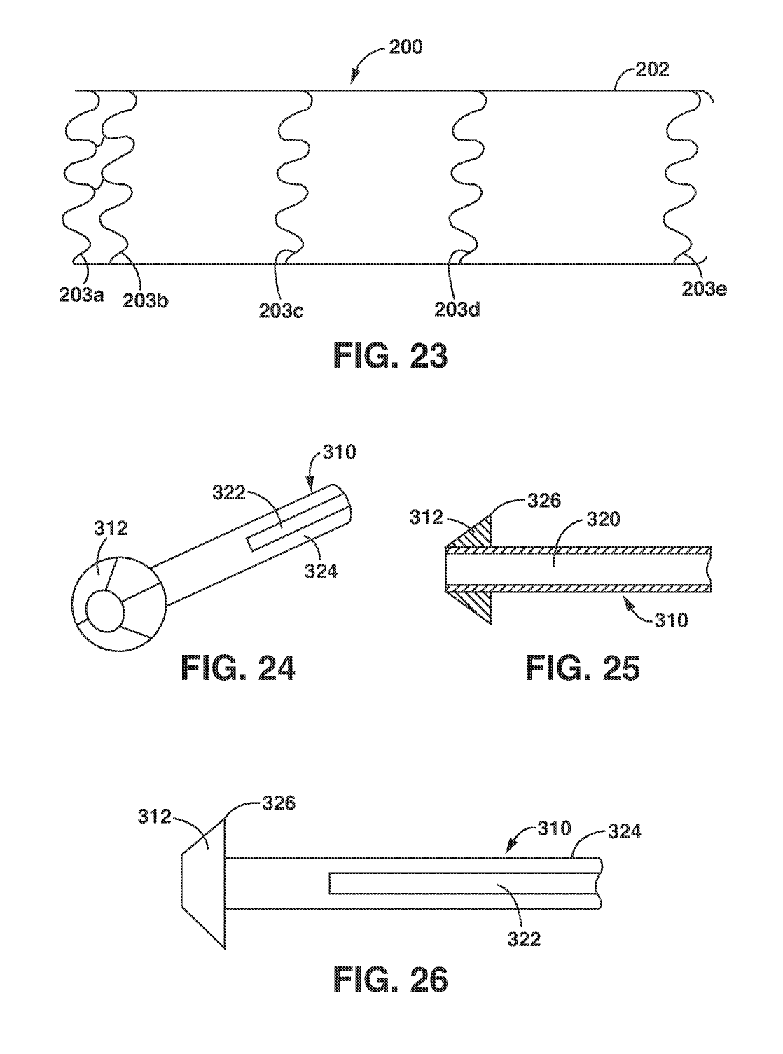

After the cutter 312 has been retracted to cut graft material 202, sheath 304 is retracted proximally relative to inner shaft 302, as shown in FIG. 22. As described above, stent stopper 308 prevents stent graft 200 from moving proximally such that sheath 304 releases stent 203e, thereby allowing stent 203e to self-expand and cause the surrounding graft material 202 to abut against the inner wall of the airway. Delivery system 300 may then be further retracted and removed from the airway, leaving stent graft 200 in place, as shown in FIG. 23.

An embodiment of a cutter assembly as mentioned and briefly described above will now be described in more detail, with reference to FIGS. 24-26. Cutter shaft 310 as shown in FIGS. 24-26 is generally a hollow tube. A proximal portion of cutter shaft 310 includes longitudinal cut-outs or notches 322 defining elongated tabs 324. Cutter shaft 310 includes a lumen 320 with a diameter sized slightly larger than an outer diameter of inner shaft 302 such that cutter shaft 310 is slidable over inner shaft 302. Cutter 312 is disposed at a distal end of cutter shaft 310. Cutter 312 is generally frustoconical in shape. An outer edge 326 of the cutter 312 is sufficiently sharp to cut graft material 202 when graft material 202 is sandwiched between cutter 312 and wedge 314, as described above.

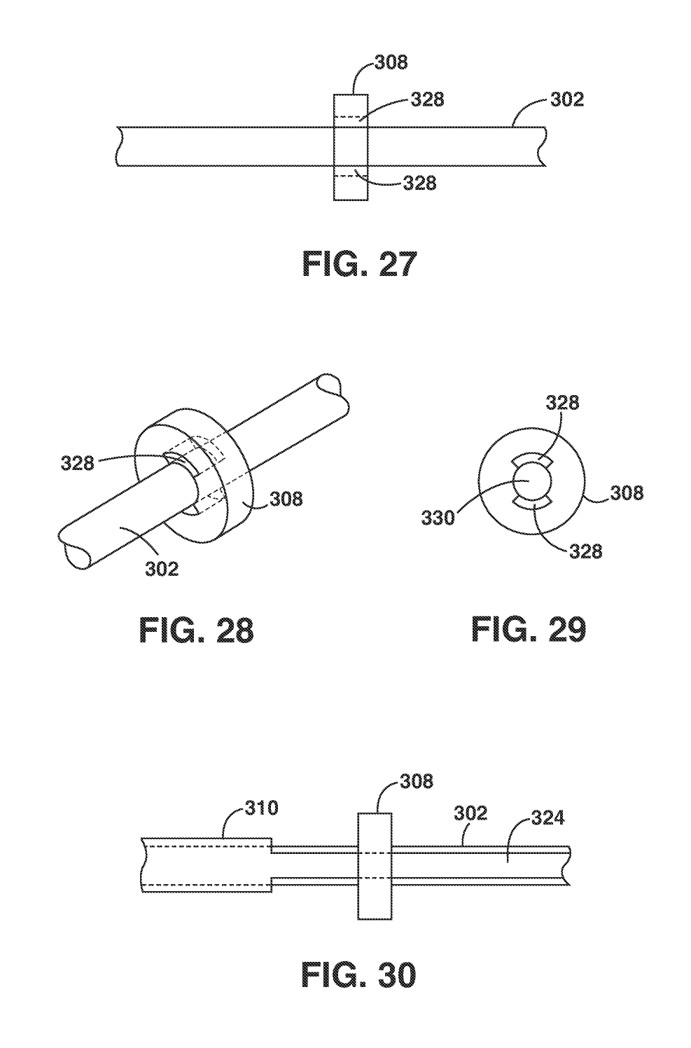

As shown in FIGS. 27-30, stent stopper 308 is fixedly attached to inner shaft 302, as by fusion, welding, adhesive, or other mechanical connections, or may be formed integrally with inner shaft 302. Stopper 308 may be a solid cylindrical disc with a central opening 330 sized to fit around inner shaft 302. Stopper 308 further includes longitudinal slots 328 sized to permit tabs 324 of cutter shaft 310 to slide therethrough, as shown in FIG. 30.

Those of ordinary skill in the art would understand that, if the stent graft 200 is already the desired length prior to implantation, then the step of cutting stent graft 200 and even providing the cutter assembly described above will not be necessary. In such a situation, the delivery system may be as described above except that the cutter shaft 310, cutter 312, and wedge 314 could be omitted. Further, the steps described above for delivering the graft 200 would be the same except that the steps involved in cutting the graft material 202 would not be necessary.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments. It will also be understood that each feature of each embodiment discussed herein, and of each reference cited herein, can be used in combination with the features of any other embodiment. Furthermore, there is no intention to be bound by any expressed or implied theory presented in the preceding technical field, background, brief summary or the detailed description. All patents and publications discussed herein are incorporated by reference herein in their entirety.

What is claimed is:

1. A stent graft comprising:
a plurality of stents substantially aligned along a common central axis;
graft material coupled to the stents such that the stents and graft material form a hollow, tubular structure including an open lumen therethrough, the graft material having an outer surface and an inner surface;
a plurality of openings disposed through the graft material; and
a plurality of one-way valves, each one-way valve associated with a corresponding opening, each valve being oriented and operable such that fluid flow is permitted from outside of the stent graft through the opening to the lumen and fluid flow is substantially prevented from the lumen through the opening to outside the stent graft.

2. The stent graft of claim 1, wherein the one way valves each comprise a flap coupled to the graft material such that the flap covers the opening.

3. The stent graft of claim 2, wherein the flap is shaped and sized to extend past a perimeter of the opening.

4. The stent graft of claim 2, wherein the flap is disposed on the inner surface of the graft material.

5. The stent graft of claim 2, wherein a first portion of the flap is attached to the inner surface of the graft material and a second portion of the flap is not attached to the inner surface of the graft material.

6. The stent graft of claim 1, wherein the graft material is a nonporous material.

7. The stent graft of claim 1, wherein the graft material is an elastic material.

8. The stent graft of claim 1, wherein the graft material is selected from the group consisting of thermoplastic elastomer, silicone, and urethane.

9. The stent graft of claim 1, wherein the fluid is air, oxygen, or a breathable mixture of gases.

10. A method of delivering a stent graft to a desired anatomic site, the method comprising the steps of:
advancing a delivery system intraluminally toward the anatomic site, wherein the delivery system includes:
an elongate inner shaft, the stent graft mounted in the delivery system around the inner shaft, the stent graft including a plurality of stents substantially aligned along a common longitudinal axis, a tubular graft material coupled to the stents, a plurality of openings disposed through the graft material, and a one way valve disposed at each opening, wherein the one way valves are configured to permit fluid flow from outside of the stent graft through the graft material to a lumen of the stent graft and to prevent fluid flow from the lumen through the graft material to outside the stent graft, the stent graft mounted in the delivery system in a radially compressed and longitudinally compressed configuration, wherein the longitudinally compressed configuration comprises the graft material disposed longitudinally between at least some of the plurality of stents being folded, and
an elongate outer sheath enclosing the stent graft in the compressed configuration for delivery to the desired anatomic site;
upon reaching the anatomic site, retracting the outer sheath to allow a first stent of the plurality of stents within a distal portion of the stent graft to radially expand to hold graft material surrounding the first stent against a wall of the anatomic site;
retracting the delivery system to longitudinally extend the graft material between the first stent and a second stent of the plurality of stents proximal to the first stent, wherein the delivery system is retracted such that the second stent is not released from the outer sheath;
retracting the outer sheath to allow the second stent to radially expand against the walls of the anatomic site; and
repeating the steps of retracting the delivery system and retracting the sheath until the desired length of the stent graft has been released from the delivery system.

11. The method of claim 10, further comprising the step of cutting the graft material between two of the plurality of stents in situ to adjust the length of the stent graft.

12. The method of claim 11, wherein the delivery system further comprises a cutter shaft slidably disposed around the inner shaft, a cutter disposed at a distal portion of the cutter shaft, and a wedge extending from an inner surface of the outer sheath and disposed proximal to the cutter, wherein the step of cutting the graft material comprises retracting the cutter shaft proximally such that the cutter moves proximally and as the cutter passes the wedge, a portion of the graft material is captured between the cutter and the wedge to cutter the portion of the graft material.

13. The method of claim 10, wherein the distal stent portion of the stent graft comprises at least two stents with at least one connecting element connecting adjacent stents.

14. The method of claim 10, wherein the one way valves each comprise a flap coupled to the graft material such that the flap covers the opening.

15. The method of claim 14, wherein the flap is disposed on an inner surface of the graft material.

16. The stent graft of claim 14, wherein a first portion of the flap is attached to the inner surface of the graft material and a second portion of the flap is not attached to the inner surface of the graft material.

17. The method of claim 10, wherein the anatomic site is an airway of a lung, and wherein when the stent graft is implanted, the stent graft prevents air from entering branch airways covered by the one-way valves during inhalation and permits air to escape the branch airways covered by the one-way valves during exhalation.

18. The method of claim 10, wherein the anatomic site is an airway of a lung.

19. A method of treating chronic obstructive pulmonary disease comprising the step of implanting a graft into an airway in a damaged portion of a lung, wherein the graft comprises a plurality of stents substantially aligned along a common central axis, graft material coupled to the stents such that the stents and graft material form a hollow, tubular structure including an open lumen therethrough, the graft material having an outer surface and an inner surface, and a plurality of one-way valves arranged about the graft material such that air is permitted to escape from branch airways of the airway through the one-way valves into the lumen during exhalation and air is substantially prevented from entering the branch airways through the one-way valves during inhalation.

20. The method of claim 19, wherein each of the one-way valves comprises an opening disposed through the graft material and a flap coupled to the graft material such that the flap covers the opening.
